(19) United States
(12) Patent Application Publication
(10) Pub. No.: US 2024/0379192 A1
(43) Pub. Date: Nov. 14, 2024

(54) MACHINE LEARNING APPROACHES TO ENHANCE DISEASE DIAGNOSTICS

(71) Applicant: ARIZONA BOARD OF REGENTS ON BEHALF OF ARIZONA STATE UNIVERSITY, Scottsdale, AZ (US)

(72) Inventors: Neal W. Woodbury, Tempe, AZ (US); Robayet Chowdhury, Richland, WA (US); Alexander T. Taguchi, Cambridge, MA (US)

(73) Assignee: ARIZONA BOARD OF REGENTS ON BEHALF OF ARIZONA STATE UNIVERSITY, Scottsdale, AZ (US)

(21) Appl. No.: 18/691,489

(22) PCT Filed: Sep. 30, 2022
(86) PCT No.: PCT/US22/77421

§ 371 Date: Mar. 13, 2024

Related U.S. Application Data
(60) Provisional application No. 63/251,417, filed on Oct. 1, 2021.

(51) Int. Cl. G16B 40/00; G16H 50/20

(52) U.S. Cl. CPC G16B 40/00; G16H 50/20

(57) ABSTRACT

Systems and methods for using machine learning to improve disease diagnostics are provided. A method can include obtaining, using a peptide array, peptide sequence data and peptide binding values from one or more samples, wherein the peptide sequence data and the peptide binding values correspond to a plurality of conditions; for each of the one or more samples, normalizing the peptide binding values according to a median binding value of peptides associated with the peptide array; and training a regressor using dense compact representations of the peptide sequence data and peptide binding values. The method can further include providing an output of the regressor to a classifier, wherein the classifier is configured to determine whether the patient has one of the plurality of conditions based on the output of the regressor.

### CROSS-REFERENCE TO RELATED APPLICATIONS

This application claims priority to U.S. Provisional Patent Application Ser. No. 63/251,417, filed Oct. 1, 2021, the disclosure of which is incorporated herein by reference.

### BACKGROUND

From the point of view of chemical biology, the humoral immune response to infection represents a truly remarkable example of rapid molecular evolution. In a matter of days to weeks, high affinity, high specificity molecular recognition of a previously unknown target is developed, mediated by antibodies (Abs). This is far more than just passive in vivo panning of a molecular library. It is instead a very active process in which initially weakly binding ligands are identified in a very sparse representation of the total possible antibody sequence space, and these are iteratively evolved to optimize both binding and specificity by orders of magnitude. The process is initiated upon recognition of a pathogen associated antigen by some of the roughly ˜10⁹ unique, native B cells which then proliferate and differentiate into short-lived plasma cells that produce an immediate wave of low affinity serum antibodies, memory B cells which preserve the initial antibody response and germinal center B cells that undergo affinity maturation via a gradient ascent optimization in sequence space to produce high affinity, pathogen directed antibodies over a periods of days to weeks. The long-lasting plasma cells and memory B cells associated with long term immunity are derived from the germinal center B cells. Together these newly amplified low and high affinity Abs contribute to an individual's circulating antibody repertoire. Several B cell repertoire studies have reported that along with the private clones unique to an individual, there exist public clones which possess Ab variable region genes commonly used by individuals responding to the same disease. This implies that, despite the vast diversity of potential antibody responses to a pathogen, a preferred set of molecular interactions often arise that result from naïve B cell activation and affinity maturation. Thus, not only does the antibody maturation process arrive at a high affinity (i.e., a specific antibody out of an almost immeasurably large sequence space of less functional possibilities), but different instances frequently result in some of the same solutions to the same problem, something that would not happen during gradient optimization on most complex, multidimensional surfaces. Understanding the nature of the molecular recognition surface associated with a humoral immune response would help shed light on how this optimization takes place so effectively in such a large, multidimensional structure space.

Deep sequencing of individual peripheral blood B cells has enabled characterization of the responding B cell repertoire at the individual clone level and its description using evolutionary models. Understanding the molecular interactions that give rise and shape to a particular humoral immune response additionally requires a similar broad profile and descriptive models of antibody binding. Common methods to generate an antibody binding profile narrowly focus on a particular pathogen, displaying short overlapping peptides presented on microarrays or in phage display libraries generated by tiling antigens or entire proteomes. However, this approach requires a priori knowledge of previously identified antigens and/or is biased towards linear epitopes, thus limiting the information contained in the produced binding profile. Panning of phage or bacterial peptide display libraries have been coupled with next generation sequencing, have provided broader binding profiles, but have primarily been used to identify epitopes through motif analysis. Panning of such libraries focuses on enriched binders, limiting the descriptive information of low and non-binding sequences required for quantitative modeling of an antibody response.

Libraries containing diverse sequence peptides have certain distinctive advantages over protein/proteome based tiled arrays: 1) use of diverse peptides allows weak to strong binding interactions including high affinity interactions of antibodies with non-cognate peptides/mimotopes compared to the tiled arrays where high affinity interactions are observed only with cognate epitopes, 2) antibodies associated with a disease state can be recognized by random peptides with higher specificity than tiled peptides. Over the past decade, a number of studies have been published using high density diverse peptide arrays as a tool for antibody binding profiling. This “immunosignature” approach captures low to high affinity interactions, and has been shown to enable robust differentiation of more than thirty different infectious and chronic diseases. The method involves applying a small amount of diluted sample of serum to a dense array of peptides with random sequences of amino acids, typically with >100,000 distinct peptide sequences of about 10 amino acids in length. Binding of immunoglobulin G (IgG) or another circulating antibody serotype to the peptides on the array is then detected quantitatively using a fluorescently labeled secondary antibody and imaged by an array scanner. Based on the pattern of binding seen in case and control samples, statistical feature selection is performed, and classifier models can be built.

Accordingly, there is a need in the technical field for predictive models for use in disease diagnostics and other applications that demonstrate improved performance over currently available predictive models and improved robustness to data noise.

### SUMMARY

The present disclosure generally relates to the use of machine learning techniques to develop enhanced disease diagnostics. In particular, the present disclosure generally relates to the development of machine learning systems using data representing multiple different disease states or conditions.

In one embodiment, the present disclosure is directed to a method comprising: obtaining, using a peptide array, peptide sequence data and peptide binding values from one or more samples, wherein the peptide sequence data and the peptide binding values correspond to a plurality of conditions; and training a regressor using dense compact representations of the peptide sequence data and peptide binding values.

In some embodiments, the method further comprises for each of the one or more samples, normalizing the peptide binding values according to a median binding value of peptides associated with the peptide array.

In some embodiments, the regressor comprises a neural network.

In some embodiments, the method further comprises providing an output of the regressor to a classifier, wherein the classifier is configured to determine whether a patient has one of the plurality of conditions based on the output of the regressor.

In some embodiments, the classifier comprises a support vector machine.

In some embodiments, the classifier comprises a neural network.

In some embodiments, the output comprises an output layer of the regressor.

In some embodiments, the output comprises predicted values of the regressor.

In some embodiments, the method further comprises obtaining a sample from a patient; obtaining, using the peptide array, sample peptide sequence data and sample peptide binding values from the sample; providing the sample peptide sequence data and sample peptide binding values to the regressor; providing an output of the regressor to a classifier; and determining, using the classifier, whether the patient has one of the plurality of conditions based on the output from the regressor.

In some embodiments, the classifier is used in connection with a diagnostic test.

In some embodiments, the classifier is used in connection with a biosurveillance system.

In one embodiment, the present disclosure is directed to a computer system for use with peptide sequence data and peptide binding values obtained using a peptide array, the computer system comprising: a processor; and a memory coupled to the processor, the memory storing instructions that, when executed by the processor, cause the computer system to execute the methods described above.

### FIGURES

The accompanying drawings, which are incorporated in and form a part of the specification, illustrate the embodiments of the invention and together with the written description serve to explain the principles, characteristics, and features of the invention. In the drawings:

FIG. 1A depicts a process of training a machine learning system, in accordance with an embodiment.

FIG. 1B depicts a machine learning system developed according to the process shown in FIG. 1A, in accordance with an embodiment.

FIG. 2A depicts shows the cohort average serum IgG binding intensity distributions for the peptide sequences, in accordance with an embodiment.

FIG. 2B depicts the number of distinguishing peptides in each cohort comparison, in accordance with an embodiment.

FIG. 3A depicts a scatter plot comparing the predicted and measured values from a neural network model fitting all of the samples simultaneously, in accordance with an embodiment.

FIG. 3B depicts the correlation coefficient of each individual sample for the simultaneous fit for the validation dataset, in accordance with an embodiment.

FIG. 3C depicts a direct comparison of the measured vs. binding correlation of each sample using the simultaneous and individual model approaches, in accordance with an embodiment.

FIG. 3D depicts the rate at which the loss function drops during training using the simultaneous fit approach, in accordance with an embodiment.

FIG. 3E depicts the dependence of the average correlation coefficient between measured and predicted binding values for each of the sample cohorts as a function of the number of peptides used in the training, in accordance with an embodiment.

FIG. 3F depicts a similarity matrix for a simultaneous fit of all of the samples together, in accordance with an embodiment.

FIG. 4 depicts a series of matrices demonstrating the weighted average classification of cohort pairs, in accordance with an embodiment.

FIG. 5 depicts a series of matrices demonstrating a comparison between the original dataset and each of the modified datasets shown in FIG. 4 using the final weight matrix as a condensed representation of the binding data, in accordance with an embodiment.

FIGS. 6A-6E depicts various aspects of discriminating between cohorts. (A) The data from the original array was analyzed in three ways: 1) directly, 2) after training a neural network and predicting the values of the array sequences, 3) after projecting the trained neural network on a complete new set of sequences. Disease discrimination was then performed for each approach using multi-class classification or by statistically determining the number of significant peptides distinguishing each cohort comparison. (B) Multi-class classification based on a neural network (see text). Classification was performed 100 times for each dataset leaving out 20% of the samples (randomly chosen) each time. Diagonal Lines: original measured array data. Cross-Hatched: neural network model prediction of binding values for array peptide sequences. Dotted: neural network projected onto a randomized set of sequences of the same overall size, composition and length distribution as the array sequences. (C) Each array element is the number of array peptides with measured binding values that are significantly higher in the sample cohort on the Y-axis compared to the sample cohort on the X-axis. Significance is defined as a p-value less than 1/N in a T-test with 95% confidence (N=122,926 total peptides, thus significant peptides have a p-value<0.05/N=4.1×10⁻⁷). (D) As in (A) except that the neural network predicted binding values of the array peptides were used instead of the measured. The mean of 10 different neural network model training runs is shown; error in the mean is <=0.3. (E) The same as in (D) except predicted values for an in silico generated array of random peptide sequences with the same average composition and length as the peptides in the array were used. The mean of 10 different sequence sets and neural network runs is shown; error of the mean is <=0.4.

FIGS. 7A-7B show the effect of added noise on multiclass classification. Noise was added to each peptide in the sample using a randomly chosen value from a gaussian distribution centered at the log₁₀ of the measured value. The sigma of the distribution was varied between 0 and 1 (the binding, and thus sigma, is on a log scale). (A) The resulting distributions of binding values for each sigma value. Distributions were determined after mean normalizing the binding values for each peptide in a cohort and then including all peptide binding values in the distribution. (B) results of applying a multi-class classifier (as in FIG. 4B) to the data for measured binding values (dashed lines) and predicted binding values (solid lines) at each value of sigma. Each classification was repeated 100 times (noise at each level was randomly added 10 times and each of these were reclassified 10 times leaving out 20% of the samples as the test set).

FIGS. 8A-8C depict the classification accuracy for high CV samples. (A) neural network predicted vs. measured values for low CV data and (B) for high CV data. (C) multiclass classification of the high CV data. Diagonal Lines, Cross-Hatched and Dotted bars represent use of measured, predicted and projected data as in FIG. 6.

### DESCRIPTION

This disclosure is not limited to the particular systems, devices and methods described, as these may vary. The terminology used in the description is for the purpose of describing the particular versions or embodiments only, and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

As used herein, “electronic neural network” or “neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., peptide sequence and binding value pair data sets or the like).

The terms “subject,” “individual,” or “patient” are used interchangeably and as used herein are intended to include human and non-human animals. Non-human animals include all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. Preferred subjects include humans in need of treatment. The methods are particularly suitable for treating humans having a disease or disorder described herein.

As used herein the terms “treat,” “treated,” or “treating” refer to both therapeutic treatment and prophylactic or preventative measures, wherein the object is to protect against (partially or wholly) or slow down (e.g., lessen or postpone the onset of) an undesired physiological condition, disorder or disease, or to obtain beneficial or desired clinical results such as partial or total restoration or inhibition in decline of a parameter, value, function or result that had or would become abnormal. For the purposes of this application, beneficial or desired clinical results include, but are not limited to, alleviation of symptoms; diminishment of the extent or vigor or rate of development of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether or not it translates to immediate lessening of actual clinical symptoms, or enhancement or improvement of the condition, disorder or disease. Treatment seeks to elicit a clinically significant response without excessive levels of side effects.

As used herein, a “sample,” such as a biological sample, is a sample obtained from a subject. As used herein, biological samples include all clinical samples including, but not limited to, cells, tissues, and bodily fluids, such as saliva, tears, breath, and blood; derivatives and fractions of blood, such as filtrates, dried blood spots, serum, and plasma; extracted galls; biopsied or surgically removed tissue, including tissues that are, for example, unfixed, frozen, fixed in formalin and/or embedded in paraffin; milk; skin scrapes; nails, skin, hair; surface washings; urine; sputum; bile; bronchoalveolar fluid; pleural fluid, peritoneal fluid; cerebrospinal fluid; prostate fluid; pus; or bone marrow. Samples include both those collected from living patients and those collected postmortem. In a particular example, a sample includes blood obtained from a subject, such as whole blood or serum. In another example, a sample includes cells collected using an oral rinse. Methods for diagnosing, predicting, assessing, and treating LD in a subject include detecting the presence or absence of antibodies to one or more biomarkers described herein, in a subject's sample. The sample may be isolated from the subject and then directly utilized in a method for determining the presence or absence of antibodies, or alternatively, the sample may be isolated and then stored (e.g., frozen) for a period of time before being subjected to analysis.

The present disclosure generally describes systems and methods for developing and implementing machine learning systems, including regressor and classifiers, configured to model correlations between peptide binding data and a variety of different conditions. Conventionally, a machine learning system used for diagnostics and related applications is trained on data related to a single condition and, accordingly, to identify that specific condition on which the machine learning system has been trained. However, the systems and methods described herein differ in that the machine learning systems are trained on data (e.g., peptide data) that is associated with a number of different conditions. Importantly, the conditions on which the machine learning systems are trained need not even be particularly related to each other in any particular manner. By training the machine learning system on a data associated with a range of different conditions, the machine learning systems' performances are even improved with respect to each individual condition.

In some embodiments, the systems and methods described herein can be used as part of or in connection with an assay and/or kit for diagnosing one or more conditions. The assay and/or kit can include reagents, probes, buffers, antibodies or other agents that enhance the binding of a subject's antibodies to biomarkers, signal generating reagents (e.g., fluorescent, enzymatic, electrochemical reagents), or separation enhancing methods (e.g., electromagnetic particles, nanoparticles, or binding reagents) for the detection of a combination of two or more biomarkers indicative thereof. In some embodiments, the probe and the signal-generating reagent may be one in the same. Techniques of use in all of these methods are discussed below.

### Machine Learning Approaches to Enhance Disease Diagnostics

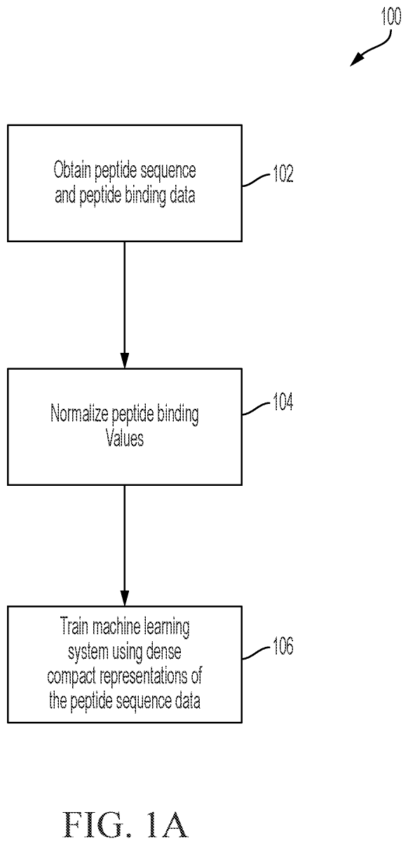

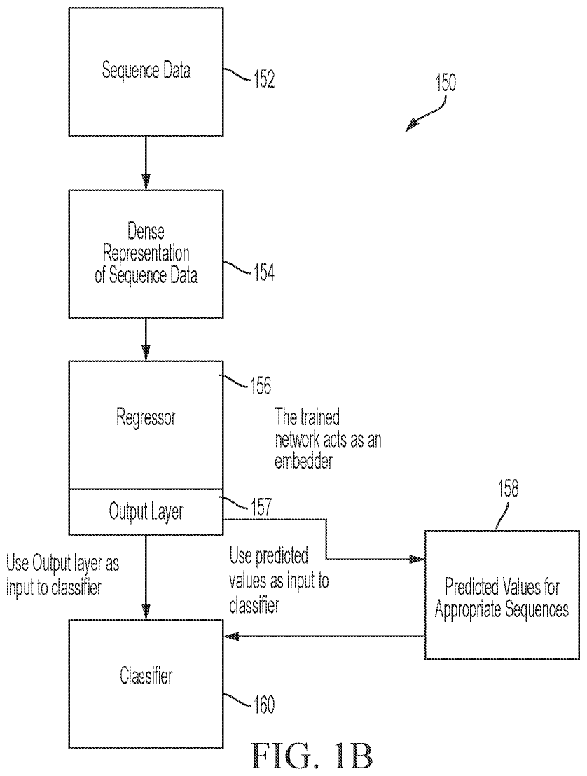

Described herein are systems and techniques for developing machine learning systems configured to identify a disease state or condition exhibited by data (e.g., peptide data) obtained from a sample from a patient. In one implementation, the systems and techniques described herein can be utilized to develop machine learning systems that model the sequence dependence of binding between peptide sequences (e.g., obtained via a peptide array) and the total serum IgG for each sample. In one embodiment, the systems and methods described can include the process 100 illustrated in FIG. 1A. The process 100 can be executed by a computer system. For example, the process 100 can be embodied as instructions stored in a memory that, when executed by a processor, cause the processor and/or computer system to perform the illustrated steps. The process 100 can be utilized in a number of different applications, including medical diagnostics, biosurveillance, or epitope discovery, as described below. Generally speaking, the process 100 involves the creation of a classification model (i.e., a classifier) that is configured to identify one or more disease states or conditions from peptide data obtained via a sample from a patient.

Accordingly, a computer system executing the process 100 can obtain 102 peptide data, such as peptide sequence data and/or peptide binding data. In one embodiment, the data can be obtained 102 via peptide arrays on one or more samples obtained from one or more patients, which may exhibit multiple disease states or conditions. The peptide data can be represented as, for example, a one-hot representation of the amino acids in each peptide sequence, i.e., the sequence can be represented as a sparse matrix of zeros and ones.

Accordingly, the computer system can normalize 104 the peptide binding values. In an embodiment where the peptide data is represented via one-hot encoding, the computer system can multiply the obtained sparse matrix representing the peptide data by an encoder matrix that linearly transforms each amino acid into a dense compact representation, i.e., a real-valued vector. In one embodiment, the resulting matrix can then be flattened to form a real-valued vector representation for a peptide sequence, which is then utilized as the input to the first hidden layer of the neural network. In some embodiments, step 104 of process 100 is not performed.

Accordingly, the computer system can train 106 a machine learning system using dense compact representations of the of the peptide sequence data. The machine learning system can include one or more neural networks, one or more support vector machines, and/or a variety of other machine learning models, for example. In some embodiments, the one or more neural networks could include a feedforward neural network. In such embodiments, the neural networks could be trained using back propagation, as is known in the technical field. In some embodiments, the machine learning system could be trained on a subset of the peptide sequence and binding paired data and the resulting machine learning system and/or individual machine learning models thereof could then be validated on the remaining subset of the peptide data, as is known in the technical field.

One embodiment of a machine learning system 150 developed using the process 100 is shown in FIG. 1B. In particular, the machine learning system 150 can include a regressor 156 that is trained on sequence data 152 and dense representations 154 of the sequence data, as described above. Once trained, the regressor 156 can function as an embedder for a classifier 160. In one embodiment, the classifier 160 could include a support vector machine. In some embodiments, classifier 160 comprises an electronic neural network. In particular, the output layer 157 and/or the predicted values 158 of the regressor 156 can be provided either individually or in combination with each other as input to a classifier 160, which then makes a classification on the provided input. In some embodiments, the input to the classifier 160 could include the predicted values 158 generated by the regressor 156 from a set of peptide sequences that between them provide differentiation between disease states. In some embodiments, the input to the classifier 160 could include the columns of the final output matrix of the regressor 156 itself, which contain a condensed version of the antibody profile information from each of the samples. In some embodiments, both types of input can be provided to the classifier 160. Accordingly, the regressor 156 can functionally obtain a broad range of information by virtue of the fact that it is trained on samples from multiple patients and multiple disease states.

One important aspect of the process 100 is that the regressor 156 is trained on peptide data that represents more than one disease state or condition. In other words, the process 100 does not train the regressor 156 only on data from a single condition and, thus, the classifier 160 is not limited to only identifying the single condition on which the machine learning system 150 was trained. Functionally, this means that the regressor 156 evaluates samples from as many patients and diseases as desired and, accordingly, generates an embedder that generally contains general knowledge about immune function and immune response to disease. The embedder can be used to generate the input provided to the classifier 160, which allows the classifier 160 to take advantage of the broad learning obtained from performing a regression on samples from many patients and with multiple diseases. As discussed in further detail below, by training the regressor 156 on data representing multiple disease states or conditions, the performance of the classifier 160 is improved in multiple respects. First, the classification performance of the classifier 160 is improved across the entire range of disease states or conditions on which the regressor 156 was trained. Second, the classifier 160 demonstrates an improved robustness to noise (e.g., Gaussian noise) in the peptide data. Third, the regressor 156 learns relationships between the various disease states or conditions that are applicable to additional disease states or conditions, which could in turn be used to improve the performance of the classifier 160 on new, unseen disease and thereby allows the classifier 160 to potentially be used to identify additional disease states or conditions on which the classifier was not trained.

In some embodiments, the classifier trained 106 as described above can subsequently be used to identify a disease state or condition exhibited by a new sample from a patient. In some embodiments, the classifier could be used to identify the presence of the disease states or conditions on which the classifier was trained. In other embodiments, the classifier could be used to identify the presence of the disease states or conditions on which the classifier was not trained.

### Neural Network Analysis

The systems and methods described above were validated experimentally. In particular, binding patterns of each protein were recorded, and a simple feed-forward, back propagation neural network model was used to relate the amino acid sequences on the array to the binding values. Remarkably, it was possible to train the network with 90% of the sequence/binding value pairs and predict the binding of the remaining left-out sequences with accuracy equivalent to the noise in the measurement. In particular, the Pearson correlation coefficients (R) between the observed and predicted binding values were equivalent to that between measured binding values of multiple technical replicates, and in some cases as high as R=0.99. In fact, accurate binding predictions (R>0.9) for some protein targets could be achieved by training on as little as a few hundred randomly chosen sequence/binding value pairs from the array. In addition, the binding predictions were specific; the neural networks captured not only the bulk binding of individual proteins, but also the differential binding between proteins. Finally, training on weakly binding sequences effectively predicted the binding values of the strongly binding sequences on the array with binding levels 1-2 orders of magnitude greater. The key point is that a very sparse sampling of total amino acid sequence space was sufficient to describe the entire combinatorial sequence space of peptide binding with high accuracy.

What both the protein array binding results imply is that the topology of sequence space associated with protein binding is mathematically well behaved, i.e., the binding features in sequence space are broad and smooth enough to be highly predictable. Indeed, it was possible to estimate that the size of the molecular recognition “hills” of the binding profile encompassed about 107 sequences on average. Serum antibody binding is clearly a much more complex and specific system than isolated proteins as it involves a large antibody repertoire including the dominant affinity matured antibodies. The finding that the immunosignature approach can differentiate disease states suggests that the molecular recognition of the immune system may also be describable using data from a very sparse sampling of sequences, as for protein/peptide binding. If so, it should be possible to develop a comprehensive and quantitative relationship between an amino acid sequence and binding associated with the specific immune response to an infectious disease.

In these particular applications, neural network-based models were used to build quantitative relationships for the sequence-antibody binding relationship using serum samples from several infectious diseases: a set of closely related flaviviridae viruses (Dengue Fever Virus, West Nile Virus, and Hepatitis C Virus), a more distantly related hepadnaviridae virus (Hepatitis B Virus), and an extremely complex eukaryotic trypanosome (Chagas Disease, Trypanasoma cruzi). By evaluating a highly disparate or unrelated set of conditions, the ability of the systems and methods described herein to accurately capture the differential information content of the array information was thoroughly evaluated.

The peptide arrays used were produced via photolithographically directed synthesis on silicon or glass wafers using methods and instrumentation common in the electronics fabrication industry. The synthesized wafers were cut into microscope slide sized pieces, each slide containing 24 arrays. Each array is consisted of ˜123,000 unique peptide sequences that were 7-12 residues in length (excluding a GSG linker connecting the C-terminus to the surface). The peptides were synthesized using 16 (A,D,E,F,G,H,K,L,N,P,Q,R,S,V,W,Y) of the 20 natural amino acids in order to simplify the synthetic process (C and M were excluded due to complications with deprotection and disulfide bond formation, and I and T were excluded due to the similarity with V and S and to decrease the overall synthetic complexity and the number of photolithographic steps required).

Serum samples were collected from three different sources: (1) Creative Testing Solutions (CTS), Tempe, AZ; (2) Sera Care; and (3) Arizona State University (ASU) (TABLE 1). The dengue4 serum samples were collected from 2 of the above sources: 30 samples were purchased from CTS and 35 samples were purchased by Lawrence Livermore National Labs (LLNL) from Sera Care before they were donated to Center for Innovations in Medicine (CIM) in the Biodesign Institute at ASU. Uninfected samples consisted of 200 CTS samples and 18 samples from healthy volunteers at ASU. For rest of the infectious cases, all samples were purchased from CTS. All the donors were reported as asymptomatic at the time of collecting serum. The Chagas disease serum samples were from asymptomatic donors and were tested as seropositive in a screening test (Abbott PRISM T. cruzi (Chagas) RR) based on the presence of T. cruzi specific antibodies and subsequently confirmed as T. cruzi seropositive using a confirmatory test. The confirmatory test was either a radioimmunoprecipitation (RIPA) or anti-T. cruzi enzyme immunoassay, EIA (Ortho T. cruzi EIA). WNV positive samples were identified at CTS by assaying for WNV RNA using a nucleic acid amplification (NAT) assay (Procleix® WNV Assay). The samples were also tested in an EIA (WNV Antibody (IgM/IgG) ELISA, Quest Diagnostics) to detect IgG and IgM antibodies as being acute and convalescent respectively. Samples with both antibodies detected in the EIA were further tested in a reverse transcriptase-polymerase chain reaction (RT-PCR) based assay as being in an acute phase. HBV samples were screened (ABBOTT PRISM HBsAg Assay Kit) for the detection of HBsAg, reactive samples were confirmed non-reactive for HCV and HIV RNA in a NAT (PROCLEIX ULTRIO ELITE ASSAY) and reactive in a HBV NAT assay, and finally considered as HBV positive with acute infection using a HBsAg Neutralization assay. If samples were tested negative for acute infection, then they were tested for anti-HBc antibodies in a test (Abbott PRISM HBC RR) to be considered as convalescent. In the case of HCV, a test approach similar to HBV was used with an additional test, a highly anti-HCV specific assay (recombinant immunoblot assay, RIBA) to confirm the samples as HCV positive in acute or convalescent phase. For uninfected controls, samples were tested as non-reactive in a NAT assay and hence confirmed as uninfected or healthy. DENV4 samples were assayed for anti-NS1 IgG as Dengue positive (convalescent) and the serotype was confirmed by indirect immunofluorescence test (IIFT). Serum samples were frozen at the time of collection and not thawed before received as aliquots in CIM.

TABLE 1

| | SAM- | SAMPLES | SAM- | GE- |
|---|---|---|---|---|
| | PLE | COLLECT- | PLES | NOME |
| DISEASE COHORT | SOURCE | ED | USED | SIZE |
| Dengue Virus, Serotype 4 | CTS and | 65 | 57 | 9600 |
| (DENV4) | Sera Care | | | |
| West Nile Virus (WNV) | CTS | 100 | 74 | 11,000 |
| Hepatitis C Virus (HCV) | CTS | 100 | 77 | 11,000 |
| Hepatitis B Virus (HBV) | CTS | 100 | 86 | 3200 |
| Chagas | CTS | 100 | 70 | 105M |
| Uninfected or control | CTS and | 218 | 177 | — |
| | ASU | | | |

Serum samples in 6 disease states were diluted (1:1) in glycerol and stored at −20° C. Before incubation, 2 μl of each serum sample (1:1 in glycerol) was prepared as 1:625 dilution in 625 μl incubation buffer (Phosphate buffer saline with 0.05 Tween 20, pH 7.2). The slides, each with a dimension of 25 mm×75 mm comprised of 24 identical peptide microarrays, were loaded into an ArrayIt microarray cassette (ArrayIt, San Mateo, CA). Then, 20 μl of the diluted serum (1:625) was added on a Whatman 903T Protein Saver Card. From the center (12 mm circle) of the protein card, a 6 mm circle was punched, and put on the top of each well in the cassette, and covered with an adhesive plate seal (3M, catalogue number: 55003076). Incubation of the diluted serum samples on the arrays were performed for 90 minutes at 37° C. with rotation at 6 rpm in an Agilent Rotary incubator. Then, the arrays were washed 3 times in distilled water and dried under nitrogen. A goat anti-human IgG (H+L) secondary antibody conjugated with either AlexaFluor 555 (Life Technol.) or AlexaFluor 647 (Life Technol.) was prepared in 1× PBST pH 7.2 to a final concentration of 4 nM. Following incubation with primary antibodies, secondary antibodies were added to the array, sealed with a 3M cover and incubated at 37° C. for 1 hour. Then, the slides were washed 3 times with PBST, followed by distilled water, removed from the cassette, sprayed with isopropanol and centrifuged dry under nitrogen, and scanned at 0.5 um resolution in an Innopsys Innoscan laser scanner, excitation 547 nm, emission 590 nm. Images in 16-bit TIFF format were aligned to a grid containing the ID and sequences for each peptide using GenePix Pro 6.0 (Molecular Devices, San Jose, CA). The raw binding data were provided in a tab limited text file following the GenePix Results (‘gpr’) file format.

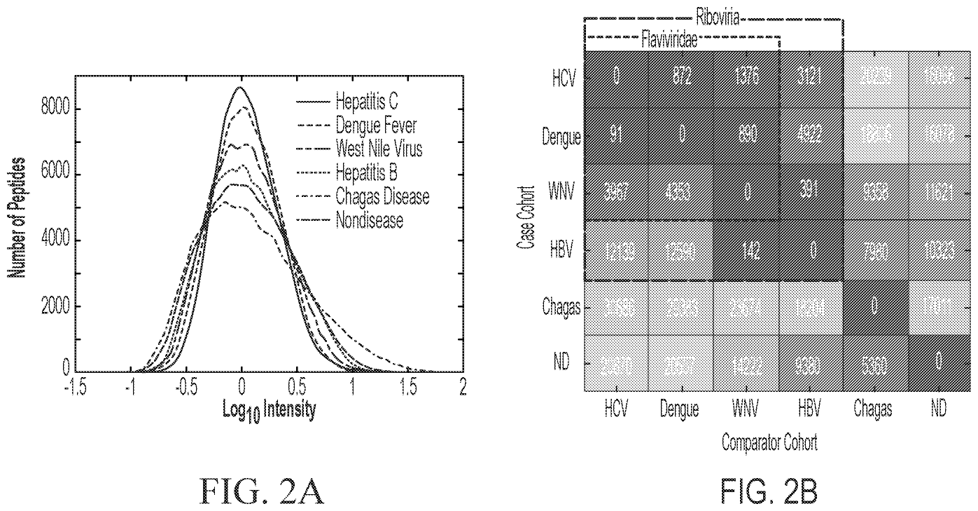

FIG. 2A shows the cohort average serum IgG binding intensity distributions of the ˜123,000 unique peptide sequences. Note that the samples were all median normalized prior to averaging within the cohort. The binding distributions are approximately log-normal and subsequent analysis of the data was performed using log₁₀ binding values, calculated as described in Methods. FIG. 2B shows the number of distinguishing peptides in each cohort comparison. In this figure, values represent the number of peptides in which the binding from the case cohort (vertical axis) is significantly greater than that from the comparator cohort (horizontal axis). Significant, in this case, means that the median binding value of that peptide in the case cohort is at least 1.5 fold higher than in the comparator cohort and the p-value associated with that peptide's binding distribution between cohorts is less than the Bonferroni cutoff (8.1×10⁻⁶).

Both the width of the distributions and the number of distinguishing peptides show a pattern. The three Flaviviruses (HCV, Dengue and WNV) have similarly sharp distributions, Hepatitis B and Chagas show distributions more similar to wild type (FIG. 2A). As one might expect, the flaviviruses are most similar to one another in terms of numbers of distinguishing peptides, but are generally more strongly distinguished from HBV (except for West Nile Virus) and very strongly distinguished from Chagas. If one follows, for example, the top row of FIG. 2B for HCV, moving to the right one sees that the numbers increase as more and more genetically dissimilar comparisons are made. West Nile virus is the exception in the figure. While it is more similar to the other flaviviruses than it is to Chagas, it is most similar, in terms of numbers of distinguishing binding signals, to HBV (FIG. 2B). This also seems to be the case with regard to the distribution of binding (FIG. 2A). While there is considerable variability in the number of distinguishing peptides in each comparison, all are sufficient to easily classify one cohort from another with near perfect accuracy, as will be discussed in more detail below.

The binding data was then utilized to train a neural network using the techniques described above and were then analyzed in two different manners. In one approach, each sample was analyzed separately so that a distinct neural network model was developed for every serum sample. In the second approach, all samples were fit together with a single neural network such that the 541 different sets of binding values were included in the same loss function. In both cases, the network consisted of two hidden layers each with 250 nodes, but in the case of individual models, the final output layer was a single column producing a single column of values, one for each peptide in the sample (hence a 123,000×1 vector). The loss function was a least squares error function based on the comparison of the predicted and measured values for the peptides in the sample. In the case of the simultaneous fit model, the final output layer consisted of 541 columns (one for each sample) and which produced 541 columns of predicted values (123,000×541 matrix). The loss function was again least squares, but based on the entire dataset.

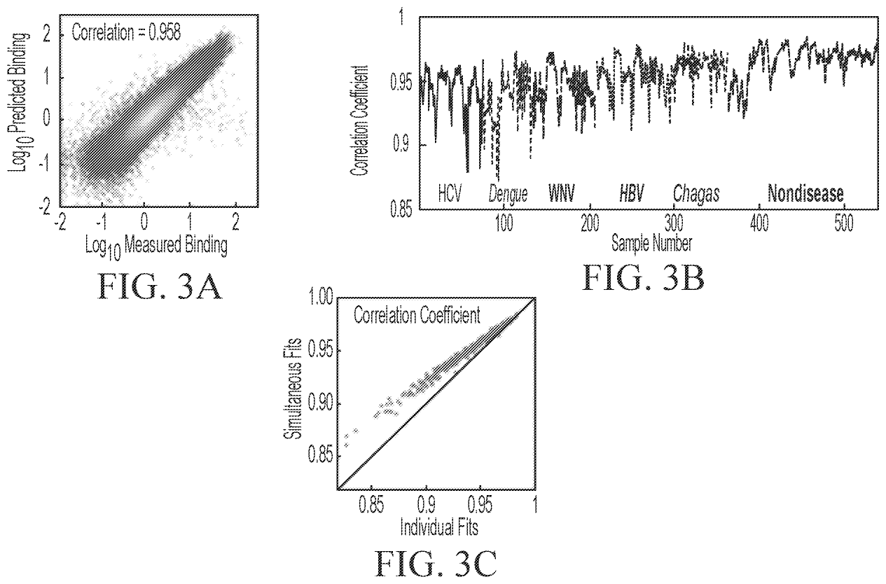

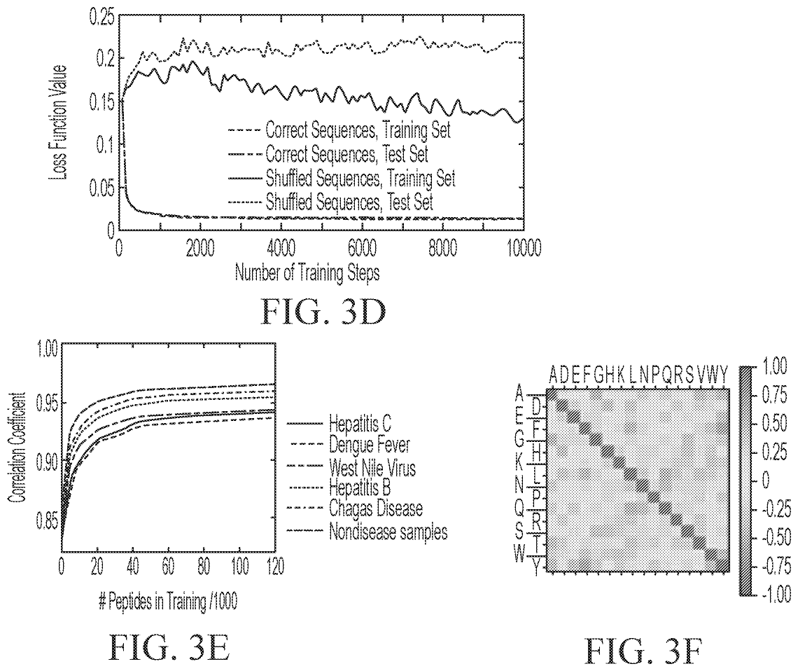

The summary of the results associated with the training of the neural network are shown in FIGS. 3A-3F. FIG. 3A shows a scatter plot comparing the predicted and measured values from a neural network model fitting all of the samples simultaneously. In this case, the neural network was trained on a training data set consisting of a random selection of 90% of the overall peptide data set. Correspondingly, the remaining 10% of the peptide data set was held out as the validation data set for the neural network. A comparison between the predicted binding values for the neural network and the actual measured binding values for the validation dataset is shown in FIG. 3A. Since the sequences used on the array are nearly randomly chosen, these sequences are representative of any randomly selected sequences in terms of model accuracy. The Pearson correlation coefficient (R) for the comparison is 0.958. Note that the correlation coefficient between measured and predicted of the other 90% of the sequences used in the training was 0.964. Thus, there is essentially no overtraining associated with the fit. FIG. 3B shows the correlation coefficient of each individual sample for the simultaneous fit for the validation dataset. The color denotes the different cohorts as indicated. Clearly some cohorts and some samples were better represented than others, but overall the vast majority of the correlation coefficients are greater than 0.9. The conclusion from this is that it is possible to create an accurate quantitative relationship between sequence and the binding of IgG for samples from all six cohorts, which should be applicable to any amino acid sequence of this size, at least on a similar array platform.

As stated above, the sequence/binding relationship for each sample was also modeled individually. FIG. 3C shows a direct comparison of the measured vs. binding correlation of each sample using the simultaneous and individual model approaches. In general, the simultaneous model is more accurate, providing a slightly, but significantly better correlation coefficient in essentially all cases.

FIG. 3D shows the rate at which the loss function drops during training using the simultaneous fit approach. As can be seen due to the fact that the top two trace referenced in the legend of FIG. 3D are essentially on top of one another, the loss function drops rapidly and in concert for the training set of sequences and the test sequences; there is little further improvement in the model after 6000 steps. An important control, which will be explored in more detail below, is to apply the neural network to data for which there is no correlation between sequence and binding. Here this was accomplished by scrambling the order of the sequences relative to the binding values. The loss function value during training is also shown for this situation in FIG. 3D. Both the training and test loss function initially rise slightly, and then the loss function for the training set of peptides slowly drops over the entire fitting process. Clearly, the neural network is directly using the sequence information in the development of the model in a very dominant way that is very different from fitting noise.

FIG. 3E explores the dependence of the average correlation coefficient between measured and predicted binding values for each of the sample cohorts as a function of the number of peptides used in the training. When at least 10,000 peptide sequences are used to train the neural network, the correlation coefficient is >0.9 and all of them are >0.85 by only 2000 peptides. It is quite remarkable that an overall binding for such a large combinatorial space can effectively be learned through such a sparse sampling of measurements in that space. That said, the correlation coefficient continues to increase throughout the increase in the size of the training set. It is clear that a more accurate and comprehensive description would be obtained if even more peptide sequences were available for training than the entire 123,000.

The data discussed above indicates that there is a learned amino acid representation that is involved in creation of a real-valued vector representing each peptide. In this case, the neural network optimizes a 10-value vector representing each amino acid. By performing dot products between these vectors and recording the cosine of the calculated angle, one can create a similarity matrix, which is shown in FIG. 3F, for a simultaneous fit of all of the samples together. Darker grey denotes similarity, blank denotes orthogonality and lighter grey denotes a situation where amino acids share similar properties but in the opposite direction. Thus, one can see that glutamic acid is similar to aspartic acid. Lysine, histidine and arginine are similar. Phenylalanine and tyrosine are similar, glutamine and asparagine are similar. Valine and leucine are similar. All of these comparisons make sense in terms of what is known about amino acids. Thus, the neural network is learning something about amino acid chemistry, though the vector descriptions optimized by the neural network are unlikely to be directly recognizable as specific physical properties commonly used.

### Learning Distinguishing Characteristics of Cohorts

While it is clear from the data described above that the neural network is able to generate an accurate relationship between sequence and the amount of serum Ab binding to peptides on the array, the fact is that only a relatively small fraction of the total Abs in serum are associated with any particular disease. In addition, because the sequences involved are not the cognate binding sequences associated with the immune response, it is not clear that the binding is specific. It would be much simpler to imagine that the neural network was recognizing generally sticky sequences, for example, rather than sequences specific to a particular immune response.

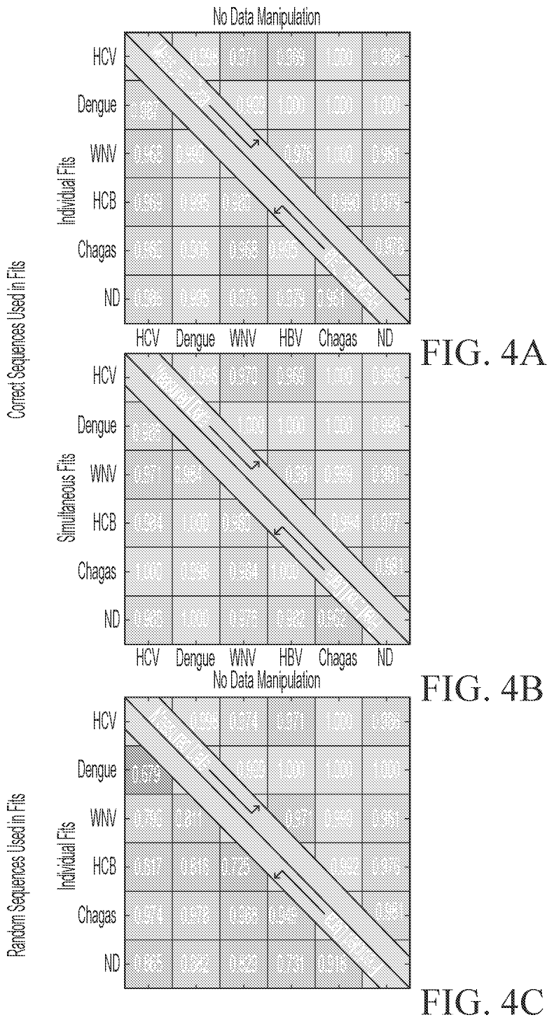

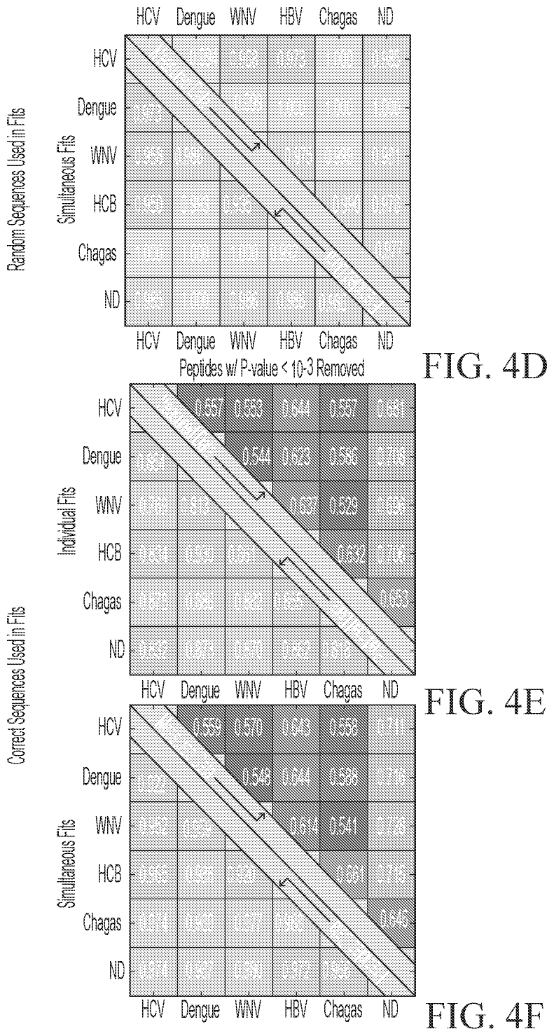

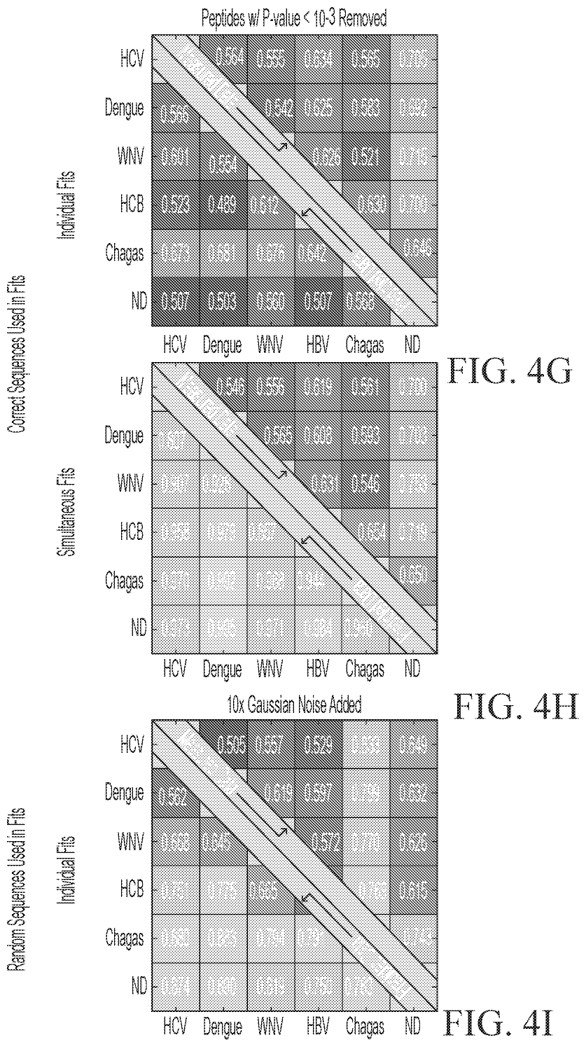

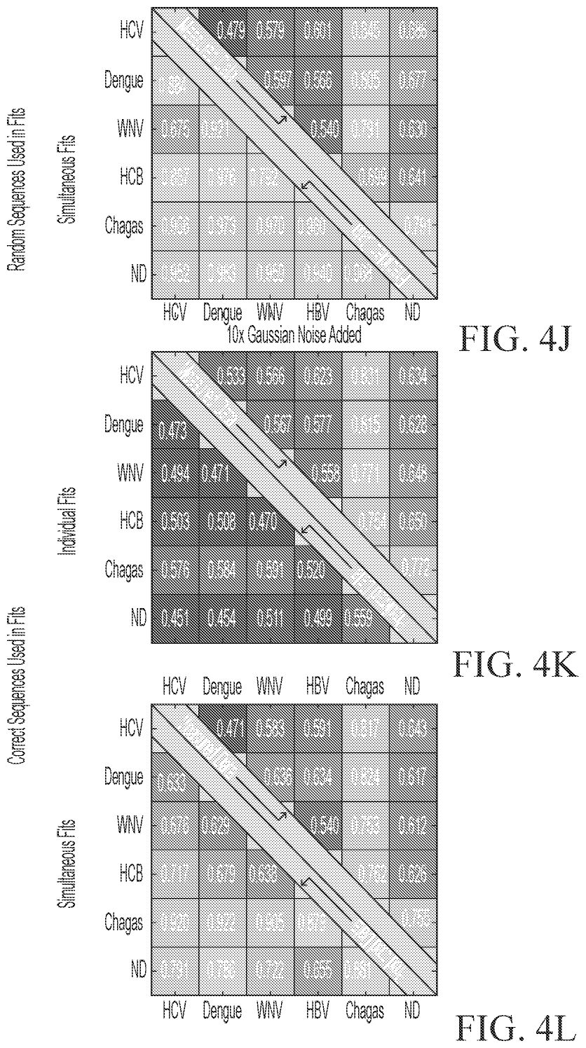

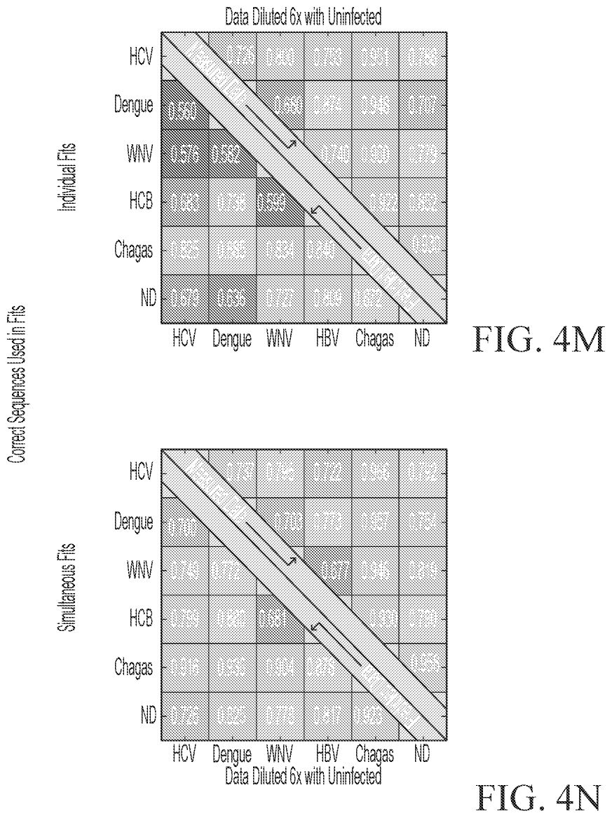

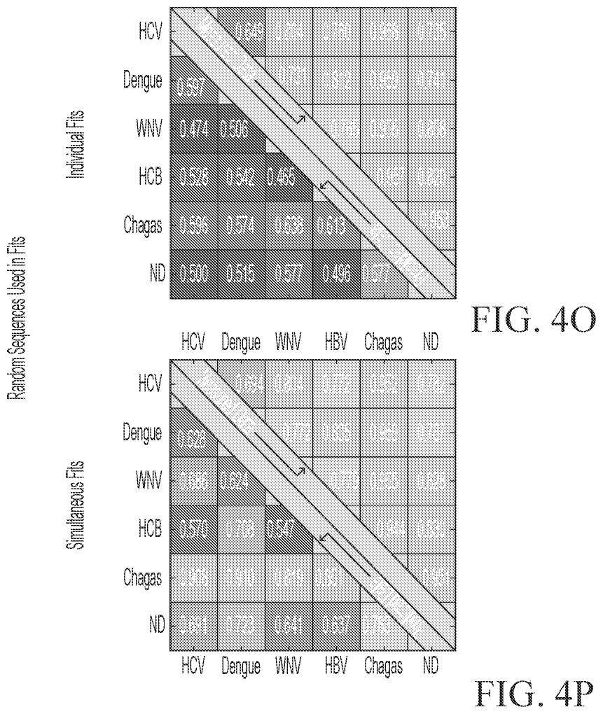

FIG. 4 explores the specificity of information captured by the neural network and its susceptibility to different kinds of noise in the binding data. In particular, FIG. 4 illustrates a series of matrices demonstrating the weighted average classification of cohort pairs. The measure of specificity being used is the weighted accuracy of a support vector machine (SVM) classifier in classifying one cohort from another using a 10-fold cross validation repeated 100 times and averaged (where “weighted” means that the accuracy of each cohort identification is determined independently and the resulting values from the two cohorts are averaged, thus removing bias due to different numbers of samples per cohort). For these classification models, the top 100 features from the array were used as determined via a 2-sided t-test between the binding values for each cohort. As an example, a value of 0.950 in FIG. 4 means that the two cohorts were classified with 95% weighted accuracy; the 10% of the samples used for validation (10× cross validation) were given the correct label by the SVM classifier 95% of the time on average after repeating the random selection of those samples and retraining of the classifier on the remaining samples 100 times.

To illustrate further, consider panel A in FIG. 4. The six cohorts are compared to one another in a 6×6 array format. The upper right half of the array represents the results with the measured data from the peptide arrays is fed directly into the classifier. Clearly, the large number of differentiating binding signals from peptides on the array (see FIGS. 2A and 2B) makes it possible to train a very accurate classifier for each cohort discrimination using the measured data. The lower left half of the array represents the same classification process applied to data generated from the neural network model. For this figure, the neural network model was used to predict values of sequences in an entirely in silico generated array of randomized sequences. Thus, the values for these sequences are not biased by training of the network on the array sequences and associated measured binding, but represent a true prediction of binding values to a novel array of sequences. In this panel, the predictions were made using a neural network that was trained on each of the 541 samples (total number of samples for all cohorts) individually. What can be seen is that the neural network has generated a new array of sequences and predicted binding values with only slightly less overall accuracy than the original data. Thus, the neural network applied to individual samples appears to retain at least a substantial amount of the differentiating signal associated with each comparison.

Panel B in FIG. 4 is the same as panel A, discussed above, except that the neural network was trained against all of the 541 samples from all cohorts simultaneously as described above. Again, this was used to create an entirely new in silico array based on new peptide sequences, and the classifier was applied to that. In this case, there is essentially no difference in the classification accuracy of the upper right (which again is the SVM model applied to the measured data directly) and the lower left (the SVM model applied to the in silico predicted array).

Panels C and D in FIG. 4 are identical to panels A and B, except that the order of the sequences on the array was randomized before training the neural network. In other words, the sequences and binding values did not correspond to one another and thus the neural network could not learn anything about the true sequence/binding relationship. In the case of the individual fits, this significantly degraded the ability of the predicted array (again a completely new randomized set of sequences) to classify accurately, but did not result in a completely random outcome. This is an important point that will be explored in more detail below. If forced to do so, the neural network can represent data using random tags (unrelated sequences) and even with generating a new set of values from a new set of random tags, some of the sample specific information is maintained. This is much more apparent when all samples are modeled simultaneously (as shown in panel D). This make sense; here the neural network must learn to differentiate the samples from one another using random inputs as tags, and, once it does so, is still able to recapitulate many of the differences from data generated from a new set of random tags. Thus, there is both sequence dependent and sequence independent learning that can take place in the neural network.

Referring back to FIG. 3D, the data shown therein demonstrates that the training, when using the correct sequence/binding relationship from the array, results in rapid improvement of the loss function in both the training and test peptides, and the correlation coefficients for both the training and test peptide sequences is very similar and generally >0.9 (little overfitting). However, when the sequence order is scrambled so that the wrong sequences are paired with binding values, the neural network slowly trains on the random tags, but the loss function for the test peptides increases. Likewise, for these fits, the correlation coefficient between measured values on the array for the training set of peptides and the predicted set slowly increases during training, generally reaching correlation coefficients of 0.15-0.4, while the correlation coefficient for the test peptides is essentially zero. Thus, when sequences are available, that information is used effectively. When sequences are not correlated with binding, the neural network simple treats the sequences as random tags and learns what it can directly from the binding data.

### Neural Network Robustness to Noise

Each of the second, third, and fourth columns (from the left) encompassing panels E-P in FIG. 4 shows different ways of either removing discriminating signals from the data or adding noise. In panels E-H of the second column of FIG. 4, for each of the 15 possible comparisons between cohorts, all of the peptides for which a two-sided tests between cohorts that resulted in a p-value less than 10-3 were removed. This is very significant because, for an array with 123,000 peptides, one expects more than 100 signals with p-values less than 10-3 simply by chance. The classifier for each combination is determined using the peptides with the best 100 p-values and, therefore, many of the peptides chosen for use in creating the classifier after the best p-values are removed will represent noise. Indeed, the classifier is largely unable to distinguish cohorts using the remaining, high p-value data directly from the array (i.e., the upper-right half of each of panels E-H in FIG. 4). However, the in silico array produced from the neural network based on individual samples (i.e., the lower left of panel E) effectively recovers much of the discriminating information. Note that the data used for the classification in the lower left of panel E of FIG. 4 was generated by retraining the neural network for each cohort comparison using the same sequences and associated binding values used to perform the classification in the upper right half of that panel. As in the rest of FIG. 4, this neural network model is then used to create a new in silico array based on randomly generated sequences. The use of a neural network trained on the same reduced dataset but with all samples simultaneously does a far superior job of creating a new in silico array that is capable of classifying the cohorts accurately. Indeed, it is only marginally worse than the overall classification when the unmanipulated dataset is used. Thus, not only is the neural network able to capture information in this case, by using patterns in the data, it effectively is a statistical noise filter, ignoring random fluctuations and incorporating nonrandom ones into the model.

A key question in this regard is exactly what the network is learning. Panels G and H of FIG. 4 show what happens when the ability to use sequence information is removed by randomly changing the order of the sequence relative to the binding values on the array. In this case, a neural network model based on individual fits of the 541 samples is almost completely incapable of recovering additional discriminating information from the datasets with the low p-value peptides removed; however, the neural network models from simultaneous fitting of all samples is only modestly less capable of providing distinguishing information between cohorts. Again, the simultaneous fits, in particular, inherently learn about the sample differences even when they are using effectively random sequences as tags, rather than relating sequence to binding.

The concept that statistical noise can be effectively filtered out from array data is supported by the results shown in the third column of FIG. 4, corresponding to panels I-L. Here, the log₁₀ of each binding value is taken and used as the mean for a random number generator with a standard deviation of 1.0, effectively creating normal distribution of noise that extends 10× in each direction from the original linear binding values. Not surprisingly, the classification of this modified dataset directly is weak, with most of the comparisons yielding accuracies of less than 0.7 (as seen in the upper right half of each of panels I-L). A neural network model created based on this data, using the correct sequence/binding pairs, is able to effectively filter statistical noise out of the data. For this network, simultaneous fits are substantially superior to networks based on individual samples (as demonstrated by panels I and J). Creating neural network models based on sequences that are uncorrelated with the binding values effectively removes the ability of the neural network to improve classification based on individual fits and substantially decreases the effectiveness of the neural network based on simultaneous fits in this regard. Again, particularly in the case of neural networks involving simultaneous fits of all data, both sequence dependent and sequence independent learning are possible.

In the last column of FIG. 4, corresponding to panels M-P, a non-statistical form of noise has been added to the data. As indicated in TABLE 1, there are 177 samples from uninfected individuals involved in this study. In this case, 100 of those were set aside and not used in the training (either for the neural network or classification). Instead, for each of the other 441 samples, one of the 100 uninfected samples was randomly selected, multiplied by 6 and added to that sample. Thus, each of the 441 remaining samples were diluted by 7-fold using data from one of many inherently heterogeneous samples. This kind of noise is very different from statistical noise. As expected, when random Gaussian noise is added to the data to create the neural network models, the correlation coefficients obtained—particularly in the test peptides—are much worse than one obtains without adding noise. As expected, the network cannot predict uncorrelated noise. However, when one simply adds in data from other samples, the relationship between sequence and binding is maintained and is biologically real. Accordingly, there is no decrease in the correlation between measured and predicted data from the neural network.

In panel M of FIG. 4, the neural network created based on individual fits of each sample is unable to generate a new in silico array that works as well as directly using the modified data in the classifier. A neural network based on simultaneous fits do better in this regard, with the in silico array produced effectively matching the original modified data in classification performance (see panel N of FIG. 4). Removing the ability of the neural networks to use sequence information by randomizing the sequences involved in the training largely eliminates the ability of the neural networks based on individual sample fits to create an in silico array that distinguishes cohorts. The neural networks based on simultaneous fits of all samples captures some of the distinguishing information between cohorts, even when they cannot use sequence dependent information, but capture is significantly degraded. In general, the neural networks used in this study are more effective at capturing distinguishing information in the presence of statistical noise than they are in the presence of noise that has a biological origin.

### Binding Data Representation

As described above, in general, the neural network learns both sequence-dependent and sequence-independent information. The ability to learn sequence-independent information is particularly evident for the neural network models based on simultaneous fits. Looking at the structure of the neural network, the final step involves a weight matrix having of one column for each of the samples involved in the training of the neural network. Each of those columns contains information that allows the neural network to distinguish between the samples. Effectively, each column is a condensed version of the 123,000 peptide dataset, concentrating the distinguishing information for each sample into a small number of values. For the analyses discussed above, that number is the width of the final hidden layer, i.e., 250. These columns or vectors can thus be used as representations of the samples in a classification, largely removing the issue of feature selection. By running the neural network and then using the final weight matrix in this way, one is effectively transforming 541 datasets of 123,000 values each into 541 vectors of only 250 values (or whatever the width of the final hidden layer is set to).

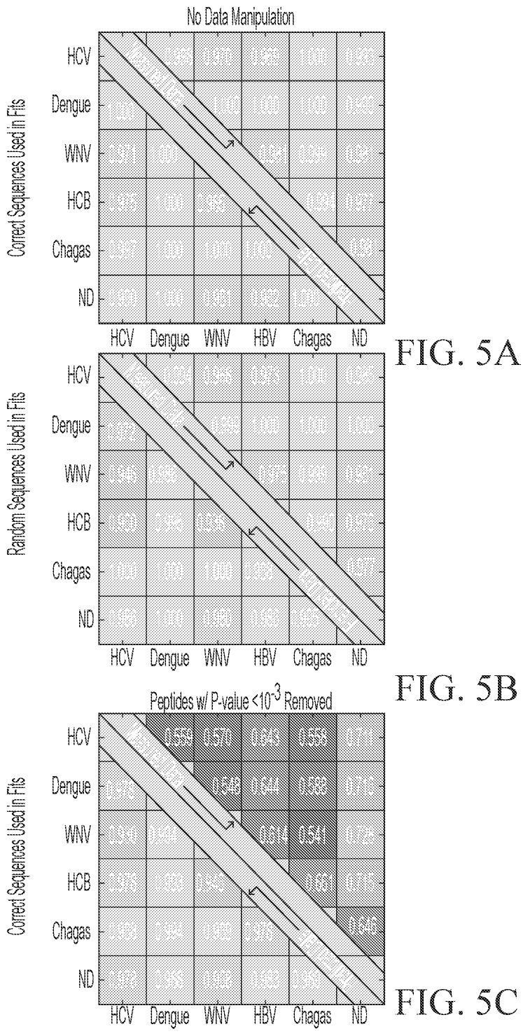

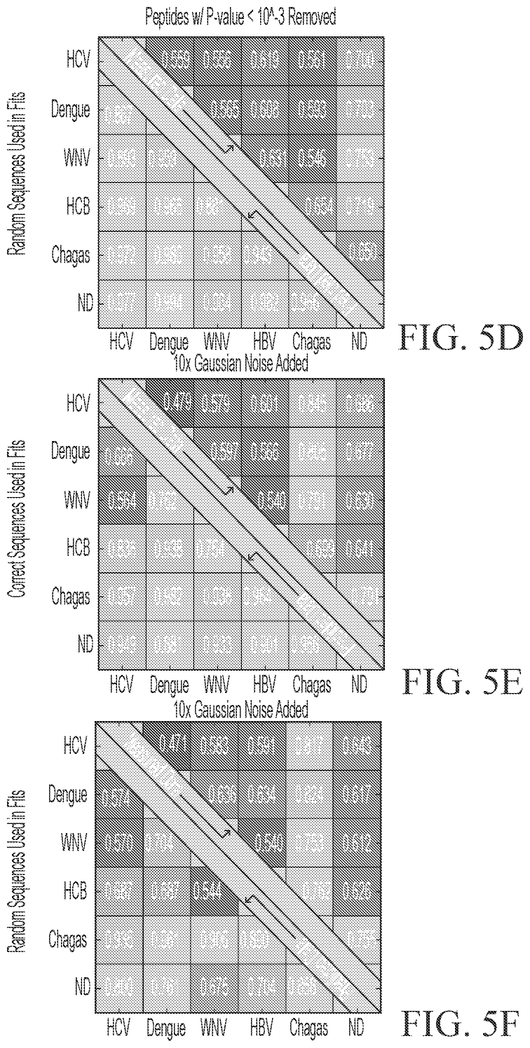

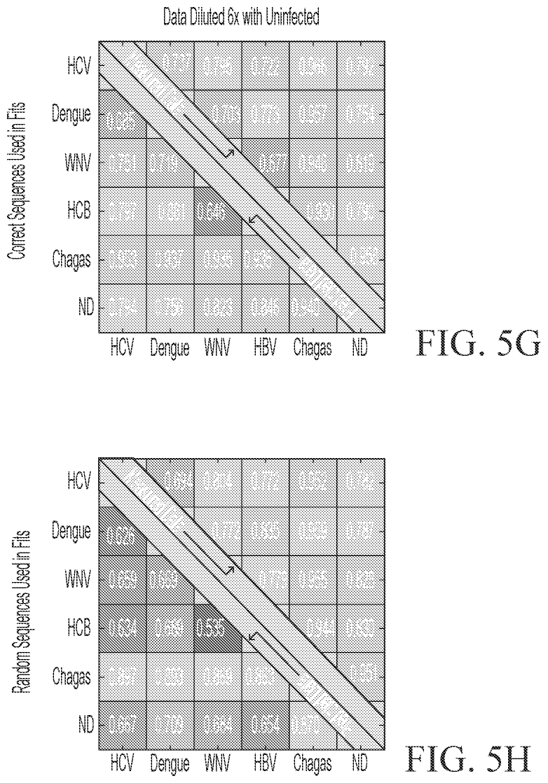

FIG. 5 shows the results of using the final weight matrix in this way for both the original dataset and each of the modified datasets described in FIG. 4. Also shown on the bottom row are the results when the neural network is trained with sequences in a random order than does not correspond to the binding data. In comparing the results shown in FIG. 5 to FIG. 4, one case see that in each case, the information captured by the neural network involved in discriminating between cohorts is largely represented in this final weight matrix. This is true for both the sequence dependent and sequence independent information. Accordingly, the final weight matrix of the trained neural network is a condensed representation of the sequence binding data.

### The Theoretical Basis of the Immunosignature Technology

Immunosignaturing technology as applied to diagnostics uses large peptide arrays as a means of capturing a quantitative immune profile to a chemically diverse set of molecules on a surface followed by a statistical analysis of the binding profile to distinguish between diseases. Because the peptide sequences involved are nearly random, the approach is essentially disease agnostic: the same peptide array platform can be used to analyze essentially any disease. By training classifier with features selected from the array, the approach has been successfully used to discriminate between serum samples from many different diseases and has been particularly effective with infectious disease, as exemplified by the near perfect ability to classify the diseases studied here. The success of the approach in this regard is remarkable. Why would antibodies that are generated by the immune system to bind tightly and specifically with pathogens show any specific interaction with arrays of random peptide sequences?

At least a partial answer to the question comes from the fact that a neural network, based on only thousands of examples of sequence/binding pairs, can generalize a sequence binding relationship such that it is able to predict the binding to essentially any peptide sequence generated in the same way and assayed under the same condition, even though the entire combinatorial sequence space involved is at least 16¹⁰ in this case or ˜10¹² (i.e., 16 amino acids and roughly 10 residues). This implies that the discriminating features on the functional surface defined by binding as a function of sequence space are very broad, encompassing up to billions of different sequences. Therefore, even an extremely sparse sampling of this sequence space contains differential binding information. Further, that surface is smooth enough so that can be accurately described by a relatively simple functional form. Presumably, only those antibodies with particularly strong and specific binding, near the top of this multidimensional structure/binding “hill,” are effective in initiating subsequent immunological activity. However, the rest of the interactions are enough to accurately define the feature and thus the differentiating interactions.

### The Nature of Information Captured in the Sequence/Binding Relationship

As described above, several different approaches were used both to create neural network models and to evaluate their information capture. In terms of an information metric, the focus was on ability to distinguish samples from one cohort from samples in another using classification methods. In most cases (e.g., as shown in FIG. 4), this meant comparing data in the measured array, or a modified version of it, with data predicted for an in silico array comprised of new sequences not on the actual array. When the unmodified dataset is used to train a neural network, either based on individual samples or via simultaneous modeling of all samples, the in silico array produced using the trained network accurately captures enough information to readily distinguish different diseases (e.g., as shown in panels A and B of FIG. 5). When the measured dataset is modified, either by removing the most significant distinguishing peptide data, by adding Gaussian noise or by diluting samples with samples from randomly selected, uninfected individuals, the neural network derived from simultaneous fits of all samples is clearly superior in terms of creating an in silico array that classifies accurately. In the case of purely statistical noise, the neural network from simultaneous modeling effectively filters out most of that noise and the in silico array produced allows one to build a far superior classifier than one could directly from the modified dataset (e.g., as shown in panels D and F of FIG. 5). Dilution of the samples analyzed by randomly selected samples of uninfected individuals is much harder to remove, but the simultaneous models result in an in silico array that recapitulates the level of information available in the modified dataset.

The fact that the neural network can effectively filter out statistical noise and allow better discrimination between cohorts means that it is bringing additional data to bear that is not available to the classifier when the dataset itself is used. This likely has three sources. First, and presumably dominantly given the loss function behavior shown by FIG. 3D, is the use of the sequence information in requiring a consistent relationship between sequence and binding. In this way, either the individual or simultaneous fits, are able to remove noise that cannot be connected to sequence information. This is most clearly seen when dealing with statistical noise. Panels E/F and I/J of FIG. 4 involve removing the statistically significant peptide data or adding Gaussian noise to that data. Both approaches result in signal swamped by statistical noise, and in both cases, the neural network is able to analyze the system and produce an in silico array with different sequences that, to a significant extent, removes statistical noise. In the case of added Gaussian noise, the predicted data correlates much less well with the measured data in combination with noise than with the measured data alone, as one would hope. This is because the network is extracting the signal that is consistent with sequence and ignoring the rest. In panels M and N of FIG. 4, the situation is different in this regard. Indeed, the neural network captures disease specific information in the arrays even when data is highly diluted by randomly selected sample data from uninfected samples, but now that “noise” is completely consistent with sequence. The neural network predicted values match the measured values in combination with the added uninfected data just as well as the measured values alone. The data is all consistent with sequence and thus not distinguishable on the basis of sequence consistency. The neural network can thus report capture the situation accurately, but it has no basis for distinguishing the real disease data from the added data in the absence of any knowledge of which samples have what disease (remembering that the neural network models are generated without any disease tags).

The second source of information captured by the neural network is only applicable to the simultaneous modeling approach. Referring back to panels E, F, I, J, M, and N of FIG. 4 (i.e., which demonstrate the introduction of different kinds of noise into the system), the neural network array predictions based on simultaneous fits always do better than individual fits at circumventing the noise. This is almost certainly partly because there is simply more data for the sequences to correlate to, thus enhancing the sequence-dependent information. In addition, however, there is at least the possibility for the neural network to learn sequence-independent information about the samples by simply comparing the 541 different binding patterns together. In other work, it may learn that when certain samples have high values for a particular peptide, others should have low values. The bottom two rows of FIG. 4 (i.e., panels C, D, G, H, K, L, O, and P) test this concept by randomizing the order of the sequences provided to the neural network relative to the binding values. This effectively removes the ability of the neural network to learn sequence-dependent relationships, but, in the case of simultaneous fits, it still remains possible to recognize patterns for certain peptides between diseases. Focusing on the samples with different kinds of random noise for the moment, what one can see is that the neural networks trained based on individual fits fail to extract significant disease dependent information from the noisy data (as shown in panels G, K, and O of FIG. 4). In contrast, the neural networks trained based on simultaneous fits extract substantial information (as shown in panels H, L, and P of FIG. 4). In this case, the sequences themselves are just tags, random inputs that the neural network uses to distinguish one set of 541 binding values from another. Even though the in silico array produced using the neural network model does not contain any of those specific sequence tags from the original array, it contains a sufficient number of new sequence tags (i.e., the same number as the physical array) such that, statistically, there will be at least some tags that predict the kinds of differential binding between samples seen in the original dataset.

The final source of disease-dependent information capture by the neural network is exemplified by panel C of FIG. 4. This is a situation where the neural network analyzed a dataset that had no added noise, and models were built for each sample individually. However, the sequence order used as input to the neural network was randomized relative to the binding values, so the neural network has no opportunity to capture sequence dependent information. Although the resulting neural network does not do a particularly good job of retaining the information from the array, it does gather some disease-dependent information. Presumably, this is because the neural network sees and must capture the overall binding characteristics of the array for each sample. FIG. 2A shows the average distributions for the different cohorts. Those distributions are distinctly different. Even though the neural network only has one sample dataset to consider at a time and cannot use sequence data to develop a consistent model, it can use the random sequences as tags to capture information about the distribution of binding values expected. A new set of random tags generated in silico has no meaning from a sequence perspective, but will recapitulate the distribution of low and high binders, and this, in itself, provides a weak, but nonzero, ability to distinguish between diseases.

### The Feature Selection Problem With Large Dataset Classification

One of the issues in dealing with many classification schemes used in disease discrimination is the need to select a set of features or biomarkers ahead of time that provide statistically significant results. Providing large amounts of non-distinguishing data (and particularly noisy data) along with distinguishing data generally results in a poorly trained model. There are many different approaches to this problem, but in situations like the one described here, where there is a relationship between biomarker structure and the signal that arises from it, using that relationship to separate signal from noise and thus find the most informative features or biomarkers can be very useful. However, in the classification approaches illustrated by FIG. 4, feature selection still had to be performed prior to classification. Simply having to pre-select features is an issue because any approach to doing so is biased in a particular direction. In FIG. 4, a t-test and resulting p-values were used. This will give features that contribute most prominently to differences in the means between two populations. This works quite well when noise is dominated by normal distributions, but in biology this is almost never the case. In reality, the system likely consists of multiple subpopulations and some features or biomarkers are better for distinguishing certain subpopulations over others.

In this respect, more holistic approaches to evaluating array based data in classifiers then picking a few features from the entire dataset would be useful. One such approach is provided by the systems and techniques described above, wherein machine learning systems (e.g., neural networks as described above) are trained on all of the samples simultaneously. In the particular implementations described above, the final matrix generated by the fit consists of 541 columns, or vectors, one for each sample used. These vectors are what take the fit information from the hidden layers and use that to distinguish the samples from one another. Thus, one would expect that essentially all the distinguishing information between samples captured by the neural network should be contained in these sample-specific vectors and condensed into much smaller number of values than the original dataset. The size of these vectors is set by the number of nodes in the final hidden layer (i.e., 250 in embodiments described above). Thus, in the particular embodiments described above, the disease-specific information from 123,000 binding values is condensed into just 250 values. As shown in FIG. 5, using the most significant 100 of these values (again, selected via 2-sided t-test), the ability to distinguish cohorts is very similar to that seen using the in silico generated arrays in shown FIG. 4. The fact that this matrix contains both the sequence-dependent and sequence-independent information captured by the neural network can be seen by comparing panels A, C, E, and G of FIG. 5 to panels A, E, I, and M of FIG. 4 (for sequence-dependent implementations) and panels B, D, F, and H of FIG. 5 to panels D, H, L, and P of FIG. 4 (for sequence-independent implementations). In that latter cases, the neural network was trained using sequences in a randomized order removing the possibility of using sequence information in the fit.

### Antibody Maturation and the Topology of the Binding Surface

Biologically, the generation of antibodies in response to infection starts with a very, very sparse sampling of an almost infinite set of possible antibody sequence variants and is followed by a maturation process that occurs through rounds of genetic changes in B cells followed by antigen-stimulated proliferation. This type of sparse sampling and gradient ascent optimization only works if two conditions are met with regard to the multidimensional binding surface encompassing antibody sequence space. First, for such sparse sampling to work at all, the functional “hills” in sequence/binding space that include the final antibody sequences must be very broad. Narrow hills would be missed entirely by sparse sampling. Second, for a gradient ascent approach to maturation to work, these hills must be locally smooth. In other words, it must be possible to climb the hill via many different paths and end up at or near the same functional capability.

The systems and techniques described above address the inverse situation. Rather than sparse sampling of the antibody sequence space probing the topology of that binding surface, sparse sampling of target sequence space was instead performed. However, one might expect the two to mirror one another. The fact that a neural network can learn to accurately and comprehensively predict antibody binding across sequence space says both that the hills or features in this binding/sequence space are very broad and that the relationship between sequence and binding is well-behaved mathematically (i.e., there are infrequent discontinuities and relatively smooth surfaces across each functional feature). FIG. 3E shows that training the neural network on even a few thousand peptide sequence/binding pairs allows it to predict binding values for sequences not used in the training with reasonable accuracy, implying that the size of the features in sequence/binding space encompass at least 10⁸-10⁹ different sequences (i.e., ˜10¹² total sequences possible and 10³-10⁴ needed for a reasonable prediction). In fact, the features are probably much broader since there likely need to be many points sampled on each feature. If the features were not smooth, local interpolation between them would not be possible, accurately predicting values of new sequences. If the sequence/binding surface of the target really does mirror in many respects that of the hypervariable regions on the antibody, then the fact that neural networks can accurately describe the binding associated with the humoral response to infectious disease makes perfect sense. In fact, it is a mathematical necessity.

### Approaches to Disease Classification and Discrimination

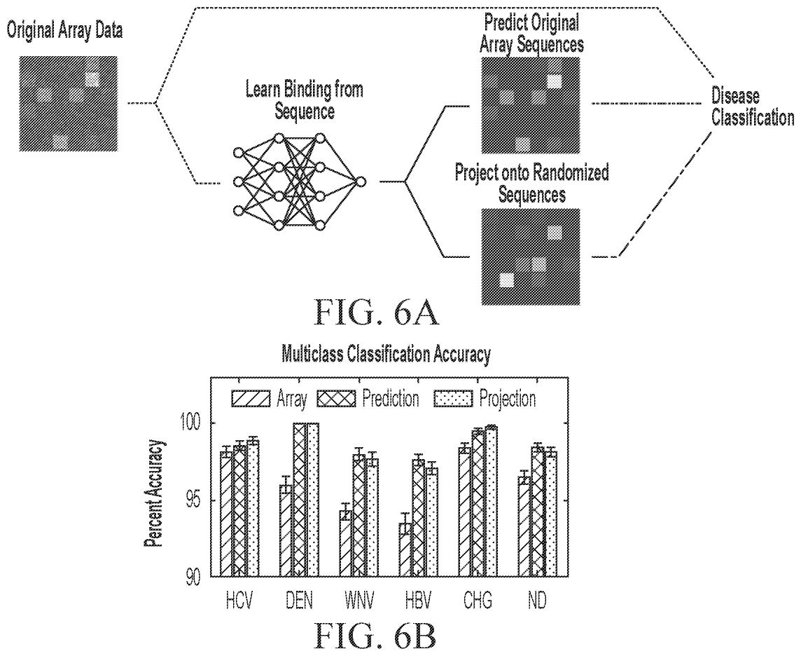

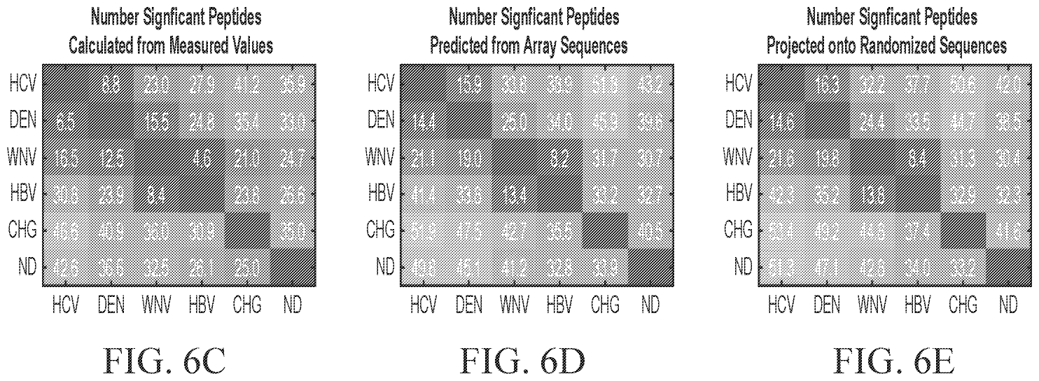

FIG. 6A is a schematic of three approaches to disease classification and discrimination. The small equally sized dashed line is the standard statistical pathway (immunosignaturing). Here no sequence information is used in the analysis and the binding values are either fed into a classifier (FIG. 6B) or used to determine number of significant peptides that distinguish diseases (FIG. 6C), as described below. Alternatively, the neural network can be used to determine a sequence/binding relationship. This relationship can either be used to recalculate predicted binding values for the array peptide sequences, forcing the data to always be consistent with the sequence (dashed line with pairs of equally sized small intervening dots), or it can be projected onto a completely new set of sequences (an in silico array, dashed line with single smaller sized intervening dashes), and those projected binding values used in classification or determining the number of significant distinguishing peptides between disease pairs.

### Values Predicted by the Neural Network Result in Better Ability to Distinguish Cohorts

In FIGS. 6C-E, the number of peptide binding values that are significantly greater in one cohort (on the Y-axis) compared to another (on the X-axis) are shown in each grid. Significance was determined by calculating p-values for each peptide in each comparison using a T-test between cohorts adjusted for multiple hypothesis comparisons using the Bonferroni correction. Significant peptides are those in which the p-value is less than 1/N (N=122,926) with a >95% confidence. FIG. 6C shows comparisons between cohorts using the measured data from the arrays. As one might expect, the sera from donors infected with the flaviviruses are most similar to one another in terms of numbers of distinguishing peptides. In general, they are more strongly distinguished from HBV (except for West Nile Virus) and very strongly distinguished from Chagas donors. If one follows, for example, the top row of FIG. 6C for HCV, moving to the right one sees that the numbers increase as more and more genetically dissimilar comparisons are made. West Nile virus is an exception in this regard. While it is more similar to the other flaviviruses than it is to Chagas, it is most similar, in terms of numbers of distinguishing peptides, to HBV (FIG. 6C).

FIG. 6D is the same as FIG. 6C except that in this case, the predicted values from the neural network model are used for the array sequences instead of the measured values. Because the network requires that a common relationship between sequence and binding be maintained for all sequences, it increases the signal to noise ratio in the system such that significantly more distinguishing peptides are identified in every comparison. The neural network was run 10 times and the results were averaged.

FIG. 6E shows results in the same format as the other two panels but using the in silico generated sequences and their binding values predicted utilizing the neural network model trained on peptide array binding data. These sequences were produced by taking the amino acids at each residue position in the original sequences and randomizing which peptide they were assigned to (considering the sequences as a matrix with rows representing peptides in the array and columns representing residue positions, order of amino acids in each column was randomized separately and at the end any spaces due to varying peptide lengths where removed). This created an in silico array with a completely new set of sequences that had the same number, overall amino acid composition and average length as the sequences on the physical array to ensure a consistent comparison. The binding values for each sample were then predicted for this in silico array and those values were used in the cohort comparisons. The number of significant peptides identified using the new sequence set (FIG. 6E) are identical to within the error for each comparison with the predictions from the actual array peptide sequences used in the training (FIG. 6D). Note that the result of generating ten different randomized in silico arrays was averaged.

Another way to understand how well distinguishing information is captured by the neural network model is to compare classification based on measured values vs. predicted values. FIG. 6B shows the result of applying a multiclass classifier, either to the measured binding values, the binding values predicted for the array sequences or binding values predicted for in silico generated sequences. A simple multiclass classifier was built using a neural network with a single hidden layer with 300 nodes. This will be referred to simply as the “multiclass classifier” to avoid confusion with the neural network used to model the sequence-binding relationship. The multiclass classifier cannot effectively use all peptides for each sample. Peptide feature selection was performed using a peptide-by-peptide T-test between the binding values of each cohort vs. all others. Either 20 features (the measured data) or 40 features (the two predicted data sets) were used per cohort, with the number of features chosen to be optimal for the dataset (see FIG. 6 legend). The training target is a one-hot representation of the sample cohort identity, and the network is set up as a regression. 80% of the samples were randomly selected and used to train the multiclass classifier and 20% were used as the test set. Each test sample was then assigned a cohort label based on the largest value in the resulting predicted output vector. The process was repeated 100 times and overall prediction accuracy determined. For every cohort, with the possible exception of HCV, classification was improved relative to direct use of the measured array values (bars with diagonal lines) when using the predicted values. This was true using either predicted values for the array sequences (bars with cross-hatched lines) or predicted values resulting from projection of the trained network on the randomized in silico array sequences (bars with dots).

### Understanding the Noise Reduction Properties of the Neural Network Modeling

The results presented above show that by using the sequence/binding information to first train a neural network model and then predicting the binding using that model (on the same or a different set of sequences), it is possible to improve the signal to noise ratio in the data, at least in terms of differentiating between disease cohorts. To understand this in more detail, the effects of added noise on the data was explored.

### Gaussian Noise Is Effectively Removed by the Model

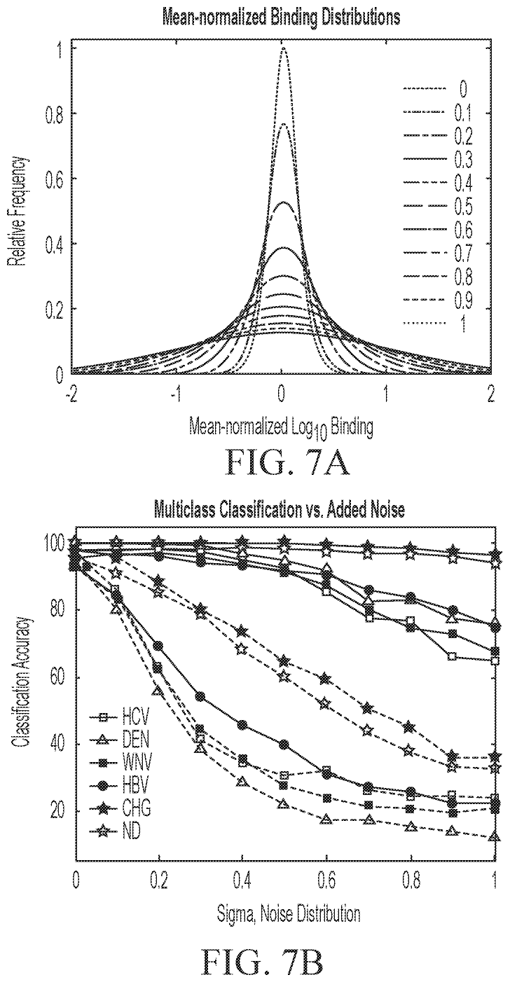

In FIG. 7, noise was artificially added to each point in the measured dataset by using a random number generator based on a gaussian distribution that was centered at the measured value:

$$f ( x ) = \frac{1}{σ \sqrt{2 π}} {e}^{- \frac{1}{2} {( \frac{x - μ}{σ} )}^{2}}$$

In the above equation, the mu (μ) is the log₁₀ of the median normalized measured binding value. Sigma (σ) was then varied from 0 to 1 to give different levels of added noise. Note that sigma=1 results in addition of noise on the order of 10-fold greater or less than the linear binding value measured (due to the log₁₀ scaling). FIG. 7A shows the resulting distribution of peptide binding values after adding noise. The peptide binding values were mean normalized across all cohorts and then plotted as a distribution, for each cohort (since this is the log₁₀ of the mean normalized value, the distributions are centered at 0). As sigma is increased, the width of the resulting distribution after adding noise increases dramatically.

FIG. 7B plots the multi-class classification accuracy of each dataset for each sample cohort as a function of sigma (this uses the same multiclass classifier as FIG. 6). The classification accuracy of the original measured data with increasing amounts of noise added drops rapidly (dashed lines). Since this is a 6-cohort multi-class classifier, random data would give an average accuracy of ˜17%. The measured values with added noise approach that accuracy level at the highest noise. However, by running the data through the neural network and then using predicted values for the same sequences as are on the array, the accuracy changes only slightly for sigma values up to about 0.5 and then drops gradually with increased noise, but always well above what would be expected for random noise. Note that a sigma of 0.5 corresponds to causing the measured values to randomly vary between about 30% and 300% of their original values.

### Neural Network Predictions of Array Signals Improved Classification of High CV Samples

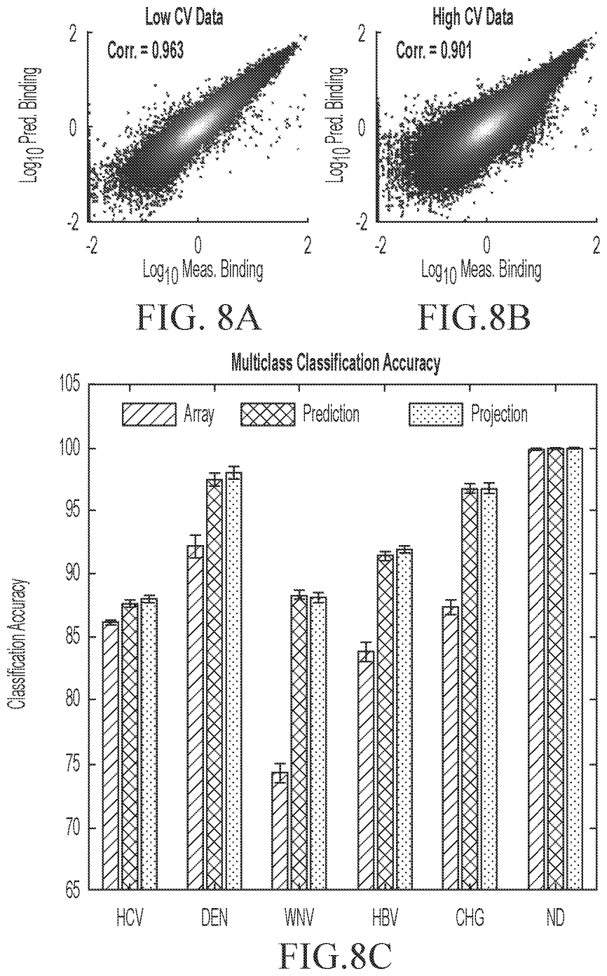

As described above, 137 samples were not used in the analyses above because they either had high CV values calculated from repeated reference sequences across the array or because there were visual artifacts such as scratches or strong overall intensity gradients across the array. A neural network model was applied to all of the 679 (542 low CV+137 high CV) samples simultaneously. Note that the model does not include any information about what cohort each sample belongs to, so modeling does not introduce a cohort bias. The overall predicted vs. measured scatter plots and correlations are given in FIG. 8A and B for both the low CV data and for the high CV data (the number of points displayed was randomly selected to be constant between datasets and make the plots comparable). Prediction of the binding values of the high CV data results in more scatter relative to measured values, due to the issues with those particular arrays.

In FIG. 8B, the measured and predicted values for the 542 low CV samples were used to train a multiclass classifier which was then used to predict the cohort class of the high CV samples. Three different data sources were used: 1) the measured array data (bars with diagonal lines), 2) predicted binding values for the array peptide sequences based on the neural network model (bars with cross-hatched lines) and 3) projected values for in silico generated arrays similar to those used in FIG. 6 (bars with dots). The classifier used was the same as that in FIG. 6 and the number of features selected was optimized for the data source as described for the analysis of FIG. 6 (20 features per cohort for the measured array data and 40 features per cohort for the two datasets based on the neural network predictions). In each case except for the non-disease samples, the use of predicted values resulted in a significantly better classification outcome.

### Uses Cases

The machine learning systems and techniques described above could be utilized in a wide variety of different applications. For example, a machine learning system developed using the techniques described above could be incorporated into a diagnostic test. In some embodiments, the machine learning systems could be used to test for one or more different diseases or conditions. In one embodiment, the machine learning systems could be developed on peptide data associated with a number of different diseases and, accordingly, the trained machine learning systems could detect the presence of those diseases or conditions in a patient sample. In one embodiment, the machine learning systems could be utilized to detect the presence of one or more diseases that were not represented in the data on which the machine learning systems were developed or trained. In one embodiment, a machine learning system developed using the techniques described above could be utilized in conjunction with a diagnostic testing kit (e.g., a peptide array). For example, a patient sample could be tested using the diagnostic kit and the data or results from the diagnostic kit could be processed through the machine learning system to identify the presence of one or more diseases or conditions exhibited thereby.

In one embodiment, a machine learning system developed using the techniques described above could be incorporated into a biosurveillance system. For example, an entity (e.g., a regulatory agency) could regularly or periodically process samples obtained from patients or other individuals to detect the presence and/or progress of a disease or condition through a given population. The samples could be obtained from, for example, blood banks.

In one embodiment, a machine learning system developed using the techniques described above could be utilized for epitope detection to inform vaccine development. In particular, the machine learning system could be utilized to as a tool to inform lead candidates for vaccine development.

Further information regarding the systems and techniques described herein can be found in Exploring the Sequence Space of Molecular Recognition Associated with the Humoral Immune Response, to Chowdhury et al., which is hereby incorporated by reference herein in its entirety.

In some embodiments, the processes and techniques described above, such as those illustrated in FIGS. 1A and 1B, can be carried out, wholly or at least partially, using a computer system. The computer system can be communicably coupled to or otherwise receive data (e.g., peptide sequence and/or peptide binding data) from machines or systems configured to analyze and/or process arrays, including the various arrays described above. The computer system can include a processor that can execute code stored on a computer-readable medium, such as a memory. The computer system may be any device that can process data and execute code that is a set of instructions to perform actions. In various embodiments, the computer system could include a database server, a web server, a desktop personal computer, a laptop personal computer, a server device, a handheld computing device, a mobile device, or combinations thereof.

In some embodiments, the processor could include a microprocessor, an application-specific integrated circuit (ASIC), a state machine, or other suitable processor. For example, the processor could include a graphics processing unit or a tensor processing unit. The processor could include one processor or any number of processors, and may access code stored in memory. The memory may be any non-transitory computer-readable medium capable of tangibly embodying code. The memory could include electronic, magnetic, or optical devices capable of providing processor with executable code. Examples of the memory include random access memory (RAM), read-only memory (ROM), a floppy disk, compact disc, digital video device, magnetic disk, an ASIC, a configured processor, or other storage device. In various embodiments, the steps of the processes described above or portions thereof can be embodied as software, hardware, firmware, or various combinations thereof.

In some embodiments, the computer system may share and/or receive data with additional components through an input/output (I/O) interface. The I/O interface could include a USB port, an Ethernet port, a serial bus interface, a parallel bus interface, a wireless connection interface, or any suitable interface capable of allowing data transfers between the computer system and another component. The additional components could include components such as an information database. In other embodiments, the computer system can include the information database.

Some further aspects are defined in the following clauses:

Clause 1: A method comprising: obtaining, using a peptide array, peptide sequence data and peptide binding values from one or more samples, wherein the peptide sequence data and the peptide binding values correspond to a plurality of conditions; and training a regressor using dense compact representations of the peptide sequence data and peptide binding values.

Clause 2: The method of Clause 1, further comprising: for each of the one or more samples, normalizing the peptide binding values according to a median binding value of peptides associated with the peptide array.

Clause 3: The method of Clause 1 or Clause 2, wherein the regressor comprises a neural network.

Clause 4: The method of any one of the preceding Clauses 1-3, further comprising: providing an output of the regressor to a classifier, wherein the classifier is configured to determine whether a patient has one of the plurality of conditions based on the output of the regressor.

Clause 5: The method of any one of the preceding Clauses 1-4, wherein the classifier comprises a support vector machine.

Clause 6: The method of any one of the preceding Clauses 1-5, wherein the classifier comprises a neural network.

Clause 7: The method of any one of the preceding Clauses 1-6, wherein the output comprises an output layer of the regressor.

Clause 8: The method of any one of the preceding Clauses 1-7, wherein the output comprises predicted values of the regressor.

Clause 9: The method of any one of the preceding Clauses 1-8, further comprising: obtaining a sample from a patient; obtaining, using the peptide array, sample peptide sequence data and sample peptide binding values from the sample; providing the sample peptide sequence data and sample peptide binding values to the regressor; providing an output of the regressor to a classifier; and determining, using the classifier, whether the patient has one of the plurality of conditions based on the output from the regressor.

Clause 10: The method of any one of the preceding Clauses 1-9, wherein the classifier is used in connection with a diagnostic test.

Clause 11: The method of any one of the preceding Clauses 1-10, wherein the classifier is used in connection with a biosurveillance system.

Clause 12: A computer system for use with peptide sequence data and peptide binding values obtained using a peptide array, the computer system comprising: a processor; and a memory coupled to the processor, the memory storing instructions that, when executed by the processor, cause the computer system to: receive the peptide sequence data and the peptide binding values corresponding to one or more samples; and train a regressor using dense compact representations of the peptide sequence data and peptide binding values.

Clause 13: The system of Clause 12, wherein the instructions that, when executed by the processor, further cause the computer system to: for each of the one or more samples, normalizing the peptide binding values according to a median binding value of peptides associated with the peptide array.

Clause 14: The system of Clause 12 or Clause 13, wherein the regressor comprises a neural network.

Clause 15: The system of any one of the preceding Clauses 12-14, wherein the instructions that, when executed by the processor, further cause the computer system to: providing an output of the regressor to a classifier, wherein the classifier is configured to determine whether a patient has one of the plurality of conditions based on the output of the regressor.

Clause 16: The system of any one of the preceding Clauses 12-15, wherein the classifier comprises a support vector machine.

Clause 17: The system of any one of the preceding Clauses 12-16, wherein the classifier comprises a neural network.

Clause 18: The system of any one of the preceding Clauses 12-17, wherein the output comprises an output layer of the regressor.

Clause 19: The system of any one of the preceding Clauses 12-18, wherein the output comprises predicted values of the regressor.

Clause 20: The system of any one of the preceding Clauses 12-19, wherein the instructions that, when executed by the processor, further cause the computer system to: providing sample peptide sequence data and sample peptide binding values obtained from a patient to the regressor; providing an output of the regressor to a classifier; and determining, using the classifier, whether the patient has one of the plurality of conditions based on the output from the regressor.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure that are within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 components refers to groups having 1, 2, or 3 components. Similarly, a group having 1-5 components refers to groups having 1, 2, 3, 4, or 5 components, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method comprising:
obtaining, using a peptide array, peptide sequence data and peptide binding values from one or more samples, wherein the peptide sequence data and the peptide binding values correspond to a plurality of conditions; and
training a regressor using dense compact representations of the peptide sequence data and peptide binding values.

2. The method of claim 1, further comprising:
for each of the one or more samples, normalizing the peptide binding values according to a median binding value of peptides associated with the peptide array.

3. The method of claim 1, wherein the regressor comprises a neural network.

4. The method of claim 1, further comprising:
providing an output of the regressor to a classifier, wherein the classifier is configured to determine whether a patient has one of the plurality of conditions based on the output of the regressor.

5. The method of claim 4, wherein the classifier comprises a support vector machine.

6. The method of claim 4, wherein the classifier comprises a neural network.

7. The method of claim 4, wherein the output comprises an output layer of the regressor.

8. The method of claim 4, wherein the output comprises predicted values of the regressor.

9. The method of claim 1, further comprising:
obtaining a sample from a patient;
obtaining, using the peptide array, sample peptide sequence data and sample peptide binding values from the sample;
providing the sample peptide sequence data and sample peptide binding values to the regressor;
providing an output of the regressor to a classifier; and
determining, using the classifier, whether the patient has one of the plurality of conditions based on the output from the regressor.

10. The method of claim 9, wherein the classifier is used in connection with a diagnostic test.

11. The method of claim 9, wherein the classifier is used in connection with a biosurveillance system.

12. A computer system for use with peptide sequence data and peptide binding values obtained using a peptide array, the computer system comprising:
a processor; and
a memory coupled to the processor, the memory storing instructions that, when executed by the processor, cause the computer system to:
receive the peptide sequence data and the peptide binding values corresponding to one or more samples; and
train a regressor using dense compact representations of the peptide sequence data and peptide binding values.

13. The computer system of claim 12, wherein the instructions that, when executed by the processor, further cause the computer system to:
for each of the one or more samples, normalizing the peptide binding values according to a median binding value of peptides associated with the peptide array.

14. The computer system of claim 12, wherein the regressor comprises a neural network.

15. The computer system of claim 12, wherein the instructions that, when executed by the processor, further cause the computer system to:
providing an output of the regressor to a classifier, wherein the classifier is configured to determine whether a patient has one of the plurality of conditions based on the output of the regressor.

16. The computer system of claim 15, wherein the classifier comprises a support vector machine.

17. The computer system of claim 15, wherein the classifier comprises a neural network.

18. The computer system of claim 15, wherein the output comprises an output layer of the regressor.

19. The computer system of claim 15, wherein the output comprises predicted values of the regressor.

20. The computer system of claim 12, wherein the instructions that, when executed by the processor, further cause the computer system to:
providing sample peptide sequence data and sample peptide binding values obtained from a patient to the regressor;
providing an output of the regressor to a classifier; and
determining, using the classifier, whether the patient has one of the plurality of conditions based on the output from the regressor.
